(19) United States
(12) Patent Application Publication
(10) Pub. No.: US 2010/0286093 A1
(43) Pub. Date: Nov. 11, 2010

(54) ANTI-INFLAMMATORY COMPOSITION CONTAINING DOCOSAHEXAENOYL LYSOPHOSPHATIDYLAMINE AS AN ACTIVE INGREDIENT

(75) Inventors: Mee Ree KIM, Yuseong-gu (KR); Dai-Eun Sok, Yuseong-gu (KR)

Correspondence Address: KLARQUIST SPARKMAN, LLP, 121 SW SALMON STREET, SUITE 1600, PORTLAND, OR, 97204, US

(73) Assignee: The Industry & Academic Cooperation in Chungnam National University (IAC)

(21) Appl. No.: 12/581,026

(22) Filed: Oct. 16, 2009

(30) Foreign Application Priority Data
May 6, 2009 (KR) 10-2009-0039141

(51) Int. Cl. A61K 31/685

(52) U.S. Cl. 514/77

(57) ABSTRACT

The present invention relates to an anti-inflammatory composition containing docosahexaenoyl lysophosphatidylamine as an active ingredient. Particularly, one of polyunsaturated lysophosphatidylcholines, docosahexaenoyl lysophosphatidylcholine inhibits intracellular NO generation mediated by lipopolysaccharide and suppresses zymosan A induced peritonitis in vivo, suggesting that it has excellent anti-inflammatory effect but has no cytotoxicity, so that it can be effectively used for prevention and treatment of inflammatory disease.

This application claims the benefit of Korea Application No. 10-2009-0039141 filed on May 6, 2009, the contents of which are hereby incorporated by reference in their entirety.

### BACKGROUND OF THE INVENTION

1. Field of the Invention

The present invention relates to a method for inhibiting inflammation using docosahexaenoyl lysophosphatidylamine and a method for preventing or treating inflammatory disease using the same.

2. Description of the Related Art

Inflammatory response is a serial, complicated physiological reaction causing enzyme activation, inflammatory mediator secretion, body fluid infiltration, cell migration and tissue destruction when tissues or cells are damaged or infected by foreign infectious agents (bacteria, fungi, virus or various allergens) to stimulate each inflammatory mediator and immune cell in peripheral blood vessel and body fluid, which carries external symptoms such as erythema, edema, fever and pain. In normal people, inflammatory response is to eliminate a foreign infectious source and to restore damaged tissues to recover normal functions in a life. But, when antigens are not completely eliminated or an endogenous material is the reason, which might induce excessive or continuous inflammatory response, the reaction rather accelerates mucosal injury and as a result it may develop disease including cancer in some cases (Jonathan Cohen, Nature, 420, 885-891, 2002).

Inflammation is mediated by diverse biochemical phenomena. In particular, nitric oxide synthase (NOS), the enzyme that generates nitric oxide (NO), and enzymes involved in prostaglandin biosynthesis are important mediators of inflammatory response. Therefore, NOS, the enzyme that generates NO from L-arginine, or cyclooxygenase (COX), the enzyme that is involved in the synthesis of prostaglandins from arachidonic acid, can be major targets of inhibiting inflammation.

According to recent studies, NOS can be classified in different groups. Expressions of brain NOS (bNOS) existing in the brain, neuronal NOS (nNOS) existing in the nervous system and endothelial NOS (eNOS) existing in the vascular system are always properly regulated. Small amount of NO generated by the said enzymes plays an important role in maintaining homeostasis but excessive NO or rapid up-regulation of NO resulted from iNOS (induced NOS) triggered by cytokines or foreign stimuli causes cytotoxicity or inflammatory responses. Chronic inflammation is known to be related to the increase of iNOS activity (Jon O. Lundberg, et al., Nature Reviews Drug Discovery, 2008). COX has two types of isoforms. COX-1 is always in cells to be involved in prostaglandin (PGs) synthesis which is necessary for cell protection. In the meantime, COX-2 is rapidly increased in cells when inflammatory response starts and plays an important role in causing inflammation. Transcription inflammatory factors including COX-2 and iNOS increasing NO and PGs are involved in causing chronic diseases such as sclerosis, Parkinson's disease, Alzheimer's disease, and colon cancer (Bengt Samuelsson, et al., Pharmacological Reviews, 59, 207-224, 2007).

Lipopolysaccharide (LPS) is an extracellular secreted bacteria toxin and induces inflammatory response and mediates the secretion of diverse inflammatory regulators such as NO, cytokine, TNF-, prostaglandin E2 and eicosanoid regulator accelerating inflammatory response (Chen Y C, et al., Biochem. Pharmacol., 61, 1417-1427, 2001). NOS is divided into two groups. cNOS is found in diverse cells (ex, nervous cells and endothelial cells) regularly and its transcription level is regulated by calcium-dependent calmodulin. In the meantime, NOS in smooth muscle cells, macrophages, hepatocytes and astrocytes is regulated by inflammatory cytokines and lipopolysaccharides. The activation of NOS plays an important role in the development of diverse inflammatory diseases by producing a large amount of NO. So, inflammation can be measured by quantifying NO induced by NOS, suggesting that it can be an index to judge inflammation progress. Specific cellular gene expressions are related to diverse inflammatory transcription factors including COX-2, iNOS, interleukin-1, interleukin-2, interleukin-6 and INF (Csaba Szabo, et al., Nature Reviews Drug Discovery, 6, 662-680. 2007).

It has recently been notified that lipid induced mediator is involved in diverse inflammation responses (asthma, rheumatoid arthritis or enteritis) (Muralikrishna Adibhatla, R., et al., Free Radic. Biol. Med., 2006; 40: 376-87; Triggiani M, et al., Biochim Biophys Acta. 1761(11): 1289-300, 2006; Matsumoto, T., et al., Curr. Med. Chem. 14, 3209-3220, 2007; Shi Y, et al., Atherosclerosis 2007; 191: 54-62).

Studies on inflammatory mediator have been undertaken, but the theory that lipid mediator is involved in inflammatory response has not been established until recent. Arachidonic acid isolated from phosphatidylcholine by PLA2 is converted into prostaglandin (PG), leukotriene (LT) and lipoxin (LX). At this time, prostaglandin and leukotriene are acting like strong inflammatory mediators (Serhan C N, et al., Nat Immunol. 6:1191-7, 2005; Farooqui A A, et al., J Neurochem. 101(3): 577-99, 2007; Funk, C. D., Science 294, 1871˜5, 2001), while lipoxin is known as a strong anti-inflammatory mediator (Schwab J M, et al., Curr Opin Pharmacol. 6(4):414-20, 2006; Kuhn H, et al., Prog Lipid Res. 2006 July; 45(4):334-56. Epub 2006 M). That is, eicosanoids generated from arachidonic acid during inflammation progress is converted into prostaglandin and leukotriene and at this time, 5-lipoxygenase is involved in leukotriene and lipoxin synthesis, while 12/15-lipoxygenase plays an important role in lipoxin synthesis (Schwab J M, et al., Curr Opin Pharmacol. 6(4):414-20, 2006). When docosahexaenoic acid is oxidized by 12/15-lipoxygenase, it metabolites into protectin D which is known as a lipid mediator having excellent anti-inflammatory activity (Serhan C N & Chiang N. Br J Pharmacol. 2008 (1); S200-215).

Lysophosphatidylcholine (lysoPC) generated during PLA2 hydrolysis shows either inflammatory or anti-inflammatory activity during inflammatory response (Fuchs B, et al., Clin. Biochem 2005; 38: 925-933; Muralikrishna Adibhatla, R., et al., Free Radic. Biol. Med. 2006; 40: 376-87; Shi Y, et al., Atherosclerosis 2007; 191: 54-62). Inflammatory lysophosphatidylcholine mediates the generation of nitric oxide (NO) or reactive oxygen species (ROS) (Colles, S. M. et al., Journal of Lipid Research 2000; 41: 1188-1198; Matsubara, M., et al., Atherosclerosis 2005; 178:57-66; Silliman C C, et al., J. Leukoc. Biol 2003; 73:511-524).

The effect of polyunsaturated lysophosphatidylcholine (lysoPC) containing polyunsaturated lipid on in vivo inflammatory response has not been clearly explained, yet. The amount of polyunsaturated lysoPC containing unsaturated lipid in animal bodies is large. For example, 1-linoleoyl lysophosphatidylcholine (linoleoyl lysoPC) and 1-arachidonoyl lysophosphatidylcholine (arachidonoyl lysoPC) are found in plasma (Okita, M. et al., Int. J. Cancer. 1997, 71, 31-34; Croset, M. et al., Biochem. J. 2000, 345, 61-67; Adachi, J. et al., Kobe. J. Med. Sci. 2006, 52, 127-140) and particularly 1-docosahexaenoyl lysophosphatidylcholine is included in major lipid in shark liver (Chen, S., et al., J. Agric. Food. Chem. 2007, 55, 9670-9677). And, lysophosphatidylcholine oxides are found in heart extract. So, lysophosphatidylcholine containing unsaturated lipid was presumed to be metabolized by lipoxygenase(LOX), which has been proved by recent studies (Huang, L. S. et al., Arch. Biochem. Biophys. 2006, 455, 119-126; Huang, L. S. et al., Lipids 2007, 42, 981-990; Huang, L. S. et al., J. Agric. Food. Chem. 2008, 56, 1224-1232). Lysophosphatidylcholine including the group consisting of linoleoyl, arachidonoyl and docosahexaenoyl is easily oxidized by reticulocyte 15-LOX or leukocyte 12/15-LOX. Even if it is strongly believed that lysophosphatidylcholine family has biological activity in vivo, physiological activity of lysophosphatidylcholine has not been disclosed.

Thus, the present inventors have been studied on physiological activity of lysophosphatidylcholine containing unsaturated lipid. As a result, the inventors confirmed that lysophosphatidylcholine containing unsaturated lipid has excellent in vivo or intracellular anti-inflammatory effect, particularly docosahexaenoyl lysophosphatidylcholine significantly inhibits NO generation, compared with other lysophosphatidylcholines, has almost no cytotoxicity, inhibits in vivo zymosan A mediated peritonitis even at a low concentration, and has excellent anti-inflammatory activity, compared with other lysophosphatidylcholines including arachidonoyl lysophosphatidylcholine. And the present inventors completed this invention by confirming that docosahexaenoyl lysophosphatidylcholine can be effectively used in preventing or treating inflammatory disease or inhibiting inflammation.

### SUMMARY OF THE INVENTION

It is an object of the present invention to provide a method for inhibiting inflammation using docosahexaenoyl lysophosphatidylamine.

It is another object of the present invention to provide a method for preventing or treating inflammatory disease using docosahexaenoyl lysophosphatidylamine.

To achieve the above objects, the present invention provides a method for inhibiting inflammation containing the step of administering docosahexaenoyl lysophosphatidylamine to a subject with inflammation.

The present invention also provides a pharmaceutical composition for preventing and treating inflammatory disease containing docosahexaenoyl lysophosphatidylamine as an active ingredient.

The present invention further provides a method for treating inflammatory disease containing the step of administering a pharmaceutically effective dose of docosahexaenoyl lysophosphatidylamine to a subject with inflammation.

The present invention also provides a method for preventing inflammatory disease containing the step of administering a pharmaceutically effective dose of docosahexaenoyl lysophosphatidylamine to a subject.

The present invention also provides health food for improvement and prevention of inflammatory disease containing docosahexaenoyl lysophosphatidylamine as an active ingredient.

The present invention also provides an anti-inflammatory external preparation for skin or nasal cavity containing docosahexaenoyl lysophosphatidylamine as an active ingredient.

In addition, the present invention provides an anti-inflammatory cosmetic composition containing docosahexaenoyl lysophosphatidylamine as an active ingredient.

### BRIEF DESCRIPTION OF THE DRAWINGS

The application of the preferred embodiments of the present invention is best understood with reference to the accompanying drawings, wherein:

FIG. 1 is a graph illustrating cell survival rate (A) and NO generation (B) in RAW 264.7 cells having inflammation induced by LPS. To investigate intracellular anti-inflammatory effect of polyunsaturated lysopolyphatidylcholine, the cells were reacted with different concentrations of polyunsaturated lysopolyphatidylcholine (linoleoyl lysophosphatidylcholine, arachidonoyl lysophosphatidylcholine and docosahexaenoyl lysophosphatidylcholine). Then, cell survival rate and NO generation were investigated (♦ linoleoyl lysophosphatidylcholine; ▪ arachidonoyl lysophosphatidylcholine; and ▴ docosahexaenoyl lysophosphatidylcholine). Each experiment was repeated three times and the results are presented by mean±standard deviation (compared with LPS treating group, *P<0.05; **P<0.01).

FIG. 2 is a graph illustrating dose-dependent in vivo anti-inflammatory effect of docosahexaenoyl lysophosphatidylcholine. Docosahexaenoyl lysophosphatidylcholine was intravenously injected into tail veins of mice and 30 min later, zymosan A was injected to induce inflammation in abdominal cavity. After administering Evans blue, plasma leakage in peritoneal fluid was measured. Each experimental group was composed of 10 mice. Results are presented by mean±standard deviation (compared with the positive control treated with zymosan A alone, *P<0.05; **P<0.01; ***P<0.001; compared with the negative control treated with saline alone, ###P<0001).

FIG. 3 is a graph illustrating dose-dependent in vivo anti-inflammatory effect of linoleoyl lysophosphatidylcholine and arachidonoyl lysophosphatidylcholine. Linoleoyl lysophosphatidylcholine (A) and arachidonoyl lysophosphatidylcholine (B) were intravenously injected into tail veins of mice and then zymosan A was injected to induce inflammation in abdominal cavity. After administering Evans blue, plasma leakage in peritoneal fluid was measured. Each experimental group was composed of 10 mice. Results are presented by mean±standard deviation (compared with the positive control treated with zymosan A alone, *P<0.05; **P<0.01; ***P<0.001; compared with the negative control treated with saline alone, ###P<0001).

FIG. 4 is a graph illustrating time-dependent in vivo anti-inflammatory effect of docosahexaenoyl lysophosphatidylcholine. Particularly, docosahexaenoyl lysophosphatidylcholine was administered to mice. Zymosan A was treated thereto at different time intervals to induce inflammation in abdominal cavity. After administering Evans blue, plasma leakage in peritoneal fluid was measured. Each experimental group was composed of 10 mice. Results are presented by mean±standard deviation (compared with the positive control treated with zymosan A alone, *P<0.05; **P<0.01; ***P<0.001; compared with the negative control treated with saline alone, ###P<0001).

FIG. 5 is a graph illustrating in vivo anti-inflammatory effect of docosahexaenoic acid and docosahexaenoyl lysophosphatidylcholine peroxide. Particularly, docosahexaenoyl lysophosphatidylcholine (1), docosahexaenoic acid (2) and 17-HPDHA lysophosphatidylcholine (3) were pretreated to mice before Zymosan A challenge. Negative and Positive indicate negative control with saline only and positive control with Zymosan A only, respectively. Zymosan A was treated thereto to induce inflammation in abdominal cavity. After administering Evans blue, plasma leakage in peritoneal fluid was measured. Each experimental group was composed of 10 mice. Results are presented by mean±standard deviation (compared with the positive control treated with zymosan A alone, *P<0.05; **P<0.01; ***P<0.001; compared with the negative control treated with saline alone, ###P<0001).

FIG. 6 is a graph illustrating dose-dependent in vivo anti-inflammatory effect of docosahexaenoyl lysophosphatidylcholine or 17-HPDHA lysophosphatidylcholine (lysophosphatidylcholine oxide), administrated intraperitoneally. Particularly, docosahexaenoyl lysophosphatidylcholine (▪) or 17-HPDHA lysophosphatidylcholine (□) was administered into abdominal cavity of mice at the concentration of 15˜150 μg/kg. 2 hours later, zymosan A was treated thereto to induce inflammation in abdominal cavity. After administering Evans blue, plasma leakage in peritoneal fluid was measured. Each experimental group was composed of 5˜10 mice. Results are presented by mean±standard deviation (compared with the positive control treated with zymosan A alone, *P<0.05; **P<0.01; ***P<0.001; compared with the negative control treated with saline alone, ###P<0001).

FIG. 7 is a graph illustrating dose-dependent in vivo anti-inflammatory effect of docosahexaenoyl lysophosphatidylethanolamine, administrated intravenously. Particularly, docosahexaenoyl lysophosphatidylethanolamine was administered into tail vein of mice at the concentration of 15˜150 μg/kg. 30 Min later, zymosan A was treated thereto to induce inflammation in abdominal cavity. After administering Evans blue, plasma leakage in peritoneal fluid was measured. Each experimental group was composed of 5-10 mice. Results are presented by mean±standard deviation (compared with the positive control treated with zymosan A alone, *P<0.05; **P<0.01; ***P<0.001; compared with the negative control treated with saline alone, ###P<0001).

### DESCRIPTION OF THE PREFERRED EMBODIMENTS

Hereinafter, terms used in this description are defined as follows.

The term “inflammation” used in this invention indicates a defensive reaction of tissue against local injury. That is, in response to harmful stressors, inflammatory response indicates biological defensive reaction to recover to original status by eliminating injury caused by the stressor. The stressor causing inflammation is infection or chemical or physical stimuli, etc. Biological constructive factors involved in inflammatory response are exemplified by low-molecular or high-molecular compounds such as free radical, protein, carbohydrate and lipid; blood plasma, hemocyte, blood vessel and connective tissue, etc. Inflammation is largely progressed into two different ways; acute and chronic inflammation. Acute inflammation is short-term reaction happening in a few days, for which blood plasma or hemocytes eliminate foreign materials through microcirculatory system. Chronic inflammation is long-term, constant inflammation, during which tissues are proliferated.

The term “prevention” used in this invention indicates every action to postpone the outbreak of inflammatory disease by administering the composition of the present invention.

The term “treatment” and “improvement” herein indicate every action to improve or relive symptoms of the disease by administering the composition of the present invention.

The term “administration” herein indicates providing the composition of the present invention to a subject by a random but proper method.

The term “subject” herein indicates any animal including human, monkey, dog, goat, pig and rat that can be administered with the composition of the present invention for the improvement of symptoms.

The term “pharmaceutically effective dose” herein indicates the amount enough to treat the disease with applicable, reasonable or risky concentration. The dose can be determined by considering many factors such as the type of inflammatory disease, severity of the disease, activity of the drug, sensitivity to the drug, administration frequency and pathway, excretion, term of treatment, co-treatment drug and other factors regarded as relevant in the medical field.

Hereinafter, the present invention is described in detail.

The present invention provides a method for inhibiting inflammation containing the step of administering docosahexaenoyl lysophosphatidylamine to a subject with inflammation.

The said docosahexaenoyl lysophosphatidylamine is selected from the group consisting of docosahexaenoyl lysophosphatidylcholine, docosahexaenoyl lysophosphatidylethanolamine and 2-docosahexaenoyl lysophosphatidylcholine.

The said docosahexaenoyl lysophosphatidylcholine is preferably represented by formula I, but not always limited thereto:

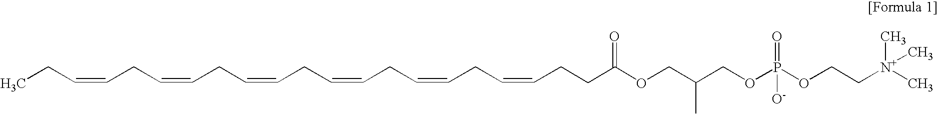

The inflammatory disease herein is preferably selected from the group consisting of peritonitis, gastritis, enteritis, arthritis, nephritis and hepatitis, and more preferably selected from the group consisting of peritonitis, enteritis and arthritis, and most preferably peritonitis, but not always limited thereto.

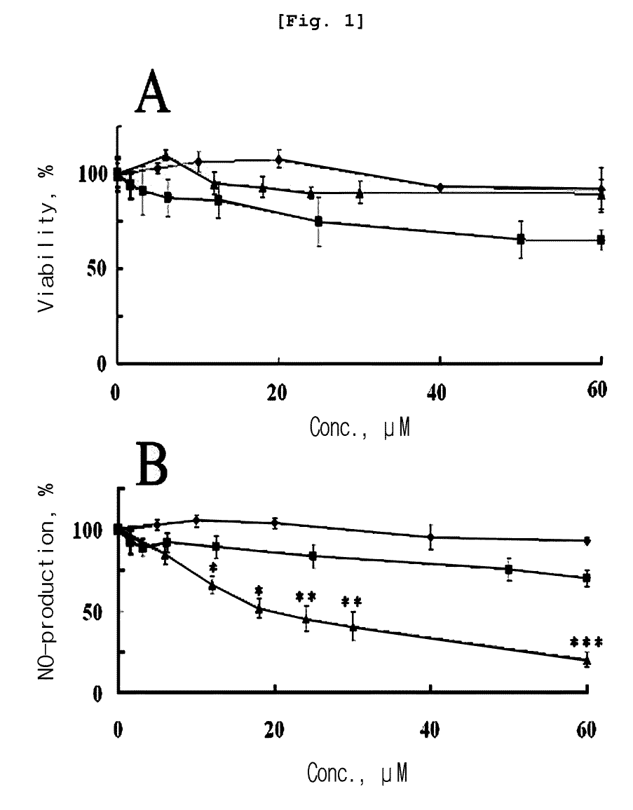

To investigate intracellular anti-inflammatory effect of lysophosphatidylcholine (lysoPC), the present inventors investigated lipopolysaccharide mediated NO generation. As a result, docosahexaenoyl lysophosphatidylcholine inhibited NO generation even at a low concentration, and the inhibitory effect was significant, compared with other lysophosphatidylcholines (see FIG. 1).

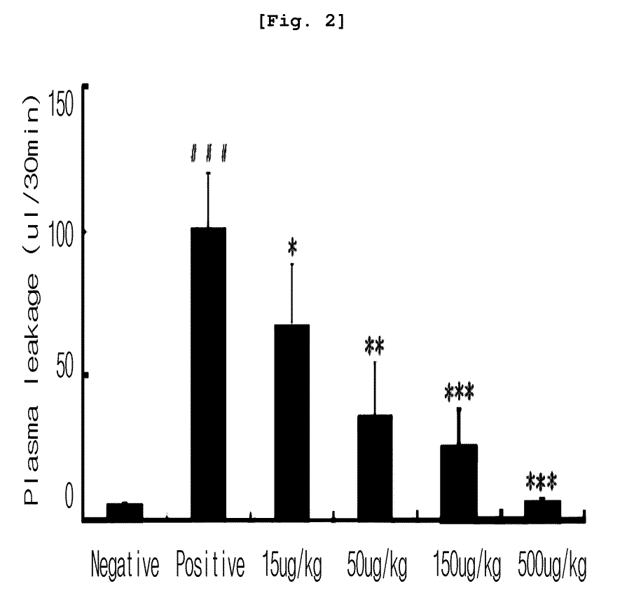

To investigate in vivo anti-inflammatory effect of docosahexaenoyl lysophosphatidylcholine, the present inventors investigated the inhibitory effect of docosahexaenoyl lysophosphatidylcholine intravenous injection on inflammation in abdominal cavity of a rat induced by zymosan. As a result, docosahexaenoyl lysophosphatidylcholine inhibited inflammation even at a low concentration, which was rather significant compared with other lysophosphatidylcholines including arachidonoyl lysophosphatidylcholine (see FIGS. 2-4).

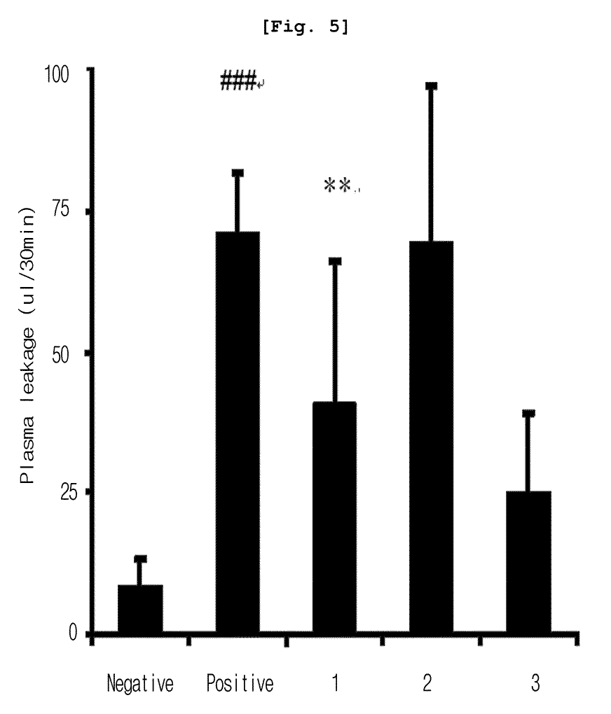

To investigate whether the anti-inflammatory effect of docosahexaenoyl lysophosphatidylcholine was its own, the present inventors investigated the inhibitory effect of the metabolites of docosahexaenoyl lysophosphatidylcholine (docosahexaenoic acid and 17-HPDHA lysophosphatidylcholine (17(S)-hydroperoxy-4,7,10,13,15,19-docosahexaenoyl lysophosphatidylcholine) on inflammation in abdominal cavity of a rat induced by zymosan A. As a result, by the intravenous injection, docosahexaenoic acid had no effect. Meanwhile, 17-HPDHA lysophosphatidylcholine had a greater anti-inflammatory effect than docosahexaenoyl lysophosphatidylcholine, suggesting that docosahexaenoyl lysophosphatidylcholine might be converted to an anti-inflammatory metabolite via 17-HPDHA lysophosphatidylcholine as an intermediate (see FIG. 5).

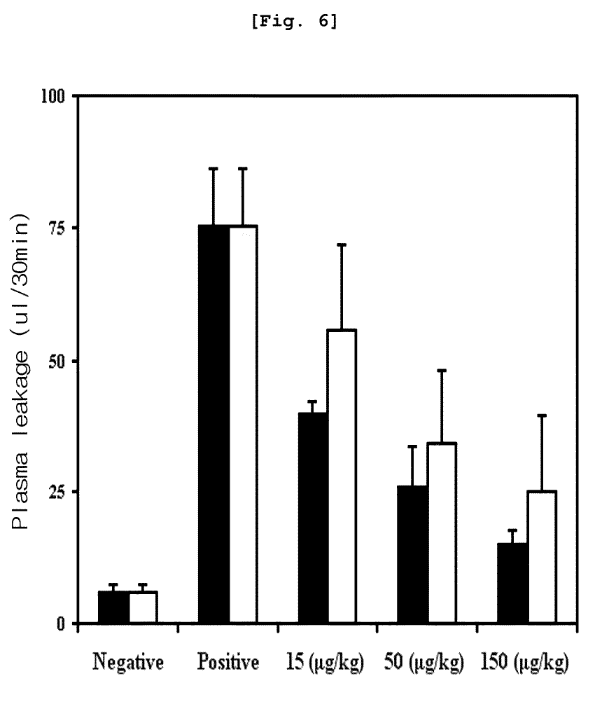

To investigate in vivo anti-inflammatory effect of docosahexaenoyl lysophosphatidylcholine, docosahexaenoyl lysophosphatidylcholine was intraperitoneally injected into a rat having abdominal inflammation induced by zymosan and then inflammation inhibitory effect was analyzed. As a result, as shown in FIG. 6, docosahexaenoyl lysophosphatidylcholine which was intraperitoneally injected demonstrated significant inhibitory effect at the concentration of 15 μg/kg or higher, and this inhibitory effect was dose-dependent up to 150 μg/kg (see FIG. 6). That is, docosahexaenoyl lysophosphatidylcholine inhibited blood plasma leakage induced by zymosan A at small doses, suggesting that it had excellent anti-inflammatory effect. Therefore, docosahexaenoyl lysophosphatidylcholine of the present invention was confirmed to have significantly higher anti-inflammatory effect than that of docosahexaenoic acid reported as an inflammation inhibitor.

Docosahexaenoyl lysophosphatidylcholine of the present invention inhibits lipopolysaccharide (LPS) mediated NO generation and suppresses peritonitis induced by zymosan A in vivo, indicating excellent anti-inflammatory effect, but has no cytotoxicity. Therefore, the administration of docosahexaenoyl lysophosphatidylcholine can be effective in the inhibition of inflammation in a subject.

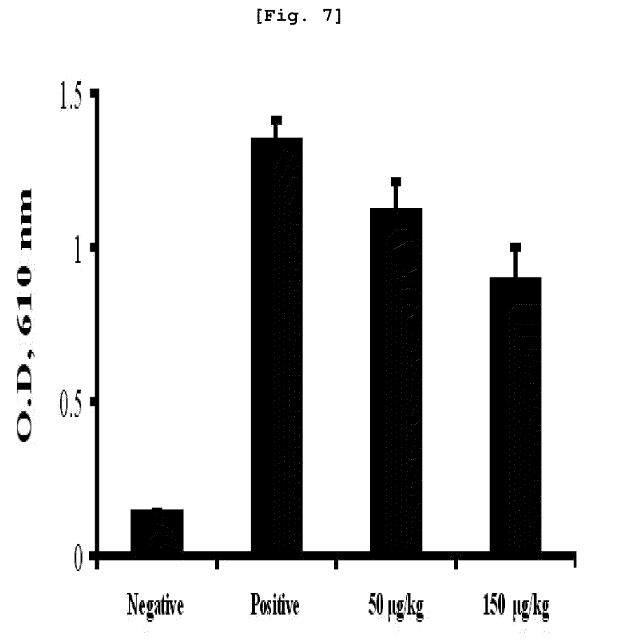

To investigate in vivo anti-inflammatory effect of docosahexaenoyl lysophosphatidylethanolamine, docosahexaenoyl lysophosphatidylethanolamine was intravenously injected into a rat having abdominal inflammation induced by zymosan A and then inflammation inhibitory effect was analyzed. As a result, as shown in FIG. 7, docosahexaenoyl lysophosphatidylethanolamine which was intravenously injected demonstrated significant inhibitory effect at the concentration of 50 μg/kg or higher, and this inhibitory effect was dose-dependent up to 150 μg/kg (see FIG. 7). That is, docosahexaenoyl lysophosphatidylethanolamine inhibited blood plasma leakage induced by zymosan A at small doses, suggesting that it had excellent anti-inflammatory effect. Therefore, docosahexaenoyl lysophosphatidylethanolamine of the present invention was confirmed to have significantly higher anti-inflammatory effect.

The anti-inflammatory composition containing docosahexaenoyl lysophosphatidylamine of the present invention can additionally include generally used carriers, excipients and diluents.

The carriers, excipients and diluents are exemplified by lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil.

The composition containing docosahexaenoyl lysophosphatidylamine of the present invention can be formulated for oral administration, for example in the forms of powders, granules, tablets, capsules, suspensions, emulsions, syrups and aerosols, and for parenteral administration, for example external use, suppositories and sterile injections, etc.

Formulations can be prepared by using general excipients or diluents such as fillers, extenders, binders, wetting agents, disintegrating agents and surfactant. Solid formulations for oral administration are tablets, pills, powders, granules and capsules. These solid formulations are prepared by mixing one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations, suppositories and injections. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

The effective dosage of docosahexaenoyl lysophosphatidylamine of the present invention can be determined according to weight and condition of a patient, severity of a disease, preparation of a drug, administration pathway and times. The effective dosage of docosahexaenoyl lysophosphatidylamine of the present invention is preferably 0.005˜0.5 mg/kg per day, and more preferably 0.015˜0.15 mg/kg per day. The administration frequency can be once a day or a few times a day. The content of docosahexaenoyl lysophosphatidylamine in the composition of the present invention is preferably 0.001˜20 weight % by the total weight of the composition.

Pharmaceutically preferable form of docosahexaenoyl lysophosphatidylamine for administration can be pharmaceutically acceptable salt thereof. And the composition can be administered independently or combined with other pharmaceutically active compounds or as a complex with other compounds.

The composition of the present invention can be administered to rats, mice, cattles and mammals including human by various pathways, for example the possible administration pathway can be oral administration, rectal administration, intravenous injection, intramuscular injection, hypodermic injection, intrauterine injection or intracerebroventricular injection.

The present invention also provides a method for treating inflammatory disease containing the step of administering a pharmaceutically effective dose of docosahexaenoyl lysophosphatidylamine to a subject with inflammatory disease.

The present invention also provides a method for preventing inflammatory disease containing the step of administering a pharmaceutically effective dose of docosahexaenoyl lysophosphatidylamine to a subject.

The said docosahexaenoyl lysophosphatidylamine is selected from the group consisting of docosahexaenoyl lysophosphatidylcholine, docosahexaenoyl lysophosphatidylethanolamine and 2-docosahexaenoyl lysophosphatidylcholine.

Docosahexaenoyl lysophosphatidylamine of the present invention inhibits inflammation in cells even at a low concentration but has no cytotoxicity. That is, docosahexaenoyl lysophosphatidylamine can be effectively used for the prevention and treatment of inflammatory disease because it can effectively inhibit inflammation in vivo even at a low concentration.

The inflammatory disease is preferably selected from the group consisting of asthma, peritonitis, gastritis, enteritis, arthritis, nephritis, hepatitis and degenerative disease, more preferably selected from the group consisting of asthma, peritonitis, enteritis and rheumatoid arthritis, and most preferably it is peritonitis, but not always limited thereto.

The composition of the present invention can include, in addition to docosahexaenoyl lysophosphatidylamine, one or more effective ingredients having the same or similar function to docosahexaenoyl lysophosphatidylamine. For the administration, the composition of the present invention can additionally include one or more pharmaceutically acceptable carriers. The pharmaceutically acceptable carrier can be selected or be prepared by mixing more than one ingredients selected from a group consisting of saline, sterilized water, Ringer's solution, buffered saline, dextrose solution, maltodextrose solution, glycerol and ethanol. Other general additives such as anti-oxidative agent, buffer solution, bacteriostatic agent, etc., can be added. In order to prepare injectable solutions such as aqueous solution, suspension and emulsion, diluents, dispersing agents, surfactants, binders and lubricants can be additionally added. The composition of the present invention can further be prepared in suitable forms for each disease or according to ingredients by following methods represented in Remington's Pharmaceutical Science (Mack Publishing Company, Easton Pa., 18th, 1990).

The composition of the present invention can be administered orally or parenterally (for example, intravenous, hypodermic, local or peritoneal injection). The effective dosage of the composition can be determined according to weight, age, gender, health condition, diet, administration frequency, administration method, excretion and severity of a disease. The dosage is 0.005˜0.05 mg/kg per day and preferably 0.015˜0.15 mg/kg per day, and administration frequency is once a day or preferably a few times a day.

The present invention also provides health food for improvement and prevention of inflammatory disease containing docosahexaenoyl lysophosphatidylamine as an active ingredient.

The said docosahexaenoyl lysophosphatidylamine is selected from the group consisting of docosahexaenoyl lysophosphatidylcholine, docosahexaenoyl lysophosphatidylethanolamine and 2-docosahexaenoyl lysophosphatidylcholine.

Docosahexaenoyl lysophosphatidylamine of the present invention inhibits intracellular NO generation derived from lipopolysaccharide and suppresses peritonitis induced by zymosan A in vivo, suggesting that it has excellent anti-inflammatory effect but no cytotoxicity, so that it can be effectively used as an active ingredient for health food for prevention and improvement of inflammatory disease.

Food to which docosahexaenoyl lysophosphatidylamine can be added is exemplified by dairy products, beverages, gums, tea, vitamins, health food, and other foods, and such food can be formulated in the forms of powders, granules, tablets, capsules or beverages.

At this time, the content of docosahexaenoyl lysophosphatidylamine in such food or beverage is 0.0001-15 weight part by the total weight of the food or beverage.

Ingredients for the health beverage composition of the present invention are not limited as long as docosahexaenoyl lysophosphatidylamine is included as an essential constituent, and any general flavors or natural carbohydrates can be included. The natural carbohydrates herein can be one of monosaccharides such as glucose and fructose, disaccharides such as maltose and sucrose, polysaccharides such as dextrin and cyclodextrin, and glucose alcohols such as xilytole, sorbitol and erythritol. Besides, natural sweetening agents such as thaumatin and stevia extract, and synthetic sweetening agents such as saccharin and aspartame can be included as a sweetening agent.

In addition to the ingredients mentioned above, the composition containing docosahexaenoyl lysophosphatidylamine of the present invention can include in variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The composition containing docosahexaenoyl lysophosphatidylamine of the present invention can also include natural fruit juice, fruit beverages and/or fruit flesh addable to vegetable beverages. All the mentioned ingredients can be added singly or together. The mixing ratio of those ingredients does not matter in fact, but in general, each can be added by 0.1-20 weight part per 100 weight part of the composition containing docosahexaenoyl lysophosphatidylamine of the present invention.

The present invention also provides an anti-inflammatory external preparation for skin or nasal cavity containing docosahexaenoyl lysophosphatidylamine as an active ingredient.

The said docosahexaenoyl lysophosphatidylamine is selected from the group consisting of docosahexaenoyl lysophosphatidylcholine, docosahexaenoyl lysophosphatidylethanolamine and 2-docosahexaenoyl lysophosphatidylcholine.

The external preparation for skin of the present invention can additionally include a supplement generally used in the field of skin science such as fatty substance, organic solvent, resolvent, concentrate, gelling agent, softener, antioxidant, suspending agent, stabilizer, foaming agent, odorant, surfactant, water, ionic or non-ionic emulsifying agent, filler, sequestering agent, chelating agent, preserving agent, vitamin, blocker, moisturing agent, essential oil, dye, pigment, hydrophilic or hydrophobic activator, lipid vesicle or other components generally used in an external preparation for skin. The amount of the above supplement can be determined as generally accepted in the field of skin science.

In addition, the present invention provides an anti-inflammatory cosmetic composition containing docosahexaenoyl lysophosphatidylamine as an active ingredient.

The said docosahexaenoyl lysophosphatidylamine is selected from the group consisting of docosahexaenoyl lysophosphatidylcholine, docosahexaenoyl lysophosphatidylethanolamine and 2-docosahexaenoyl lysophosphatidylcholine.

The cosmetic composition of the present invention can contain a composition selected from the group consisting of soluble vitamins, oil-soluble vitamins, high-molecular peptides, high-molecular polysaccharides, sphingo lipids and seaweed extracts, but not always limited thereto.

The soluble vitamin herein can be any one that can be combined with cosmetics, but preferably vitamin B1, vitamin B2, vitamin B6, pyridoxine, pyridoxine.HCl, vitamin B12, pantothenic acid, nicotinic acid, nicotinic acid amide, folic acid, vitamin C, vitamin H, etc or their salts (thiamine hydrochloride, sodium ascorbate, etc) and their derivatives (ascorbate-2-sodium phosphate, ascorbate-2-magnesium phosphate, etc) are selected, but not always limited thereto.

The oil-soluble vitamin herein can be any one that can be combined with cosmetics, but preferably vitamin A, carotene, vitamin D2, vitamin D3, vitamin E (d1-alpha tocopherol, d-alpha tocopherol, etc) and their derivatives (ascorbyl palmitate, ascorbyl stearate, ascorbyl dipalmitate, di-alpha tocopherol acetate, di-alpha tocopherol nicotinate, DL-pantothenyl alcohol, D-pantothenyl alcohol, pantothenyl ethyl ether, etc) are selected, but not always limited thereto.

The high-molecular peptide herein can be any one that can be combined with cosmetics, but preferably selected from the group consisting of collagen, hydrolyzed collagen, gelatin, elastin, hydrolyzed elastin and keratin, but not always limited thereto.

The high-molecular polysaccharide herein can be any one that can be combined with cosmetics, but preferably selected from the group consisting of hydroxyethylcellulose, xanthan gum, sodium hyaluronate, chondroitin sulfate or its salts (sodium salt, etc), but not always limited thereto.

The sphingo lipid herein can be any one that can be combined with cosmetics but preferably selected from the group consisting of ceramide, phytosphingosine, and sphingo glycolipid, but not always limited thereto.

The seaweed extract herein can be any one that can be mixed with cosmetics, but preferably selected from the group consisting of brown seaweed extract, red seaweed extract, and green seaweed extract, and carrageenan, alginic acid, sodium alginate or potassium alginate, purified from the above extracts, but not always limited thereto.

The cosmetic composition of the present invention can contain other ingredients generally used in the field of cosmetics, in addition to the above essential ingredient, which are exemplified by oil components, moisturizers, emollients, surfactants, organic and inorganic dyes, organosols, UV absorbents, antiseptics, bactericides, antioxidants, plant extracts, pH regulators, alcohols, pigments, flavors, hematogenic accelerators, anhydrotic agents and purified water, but not always limited thereto.

The oil component herein can be selected from the group consisting of ester oil, hydrocarbon oil, silicon oil, fluorinated oil, animal oil and vegetable oil, but not always limited thereto. The ester oil is exemplified by tri-2-ethylhexaneglyceryl, 2-ethylhexanecetyl, isopropyl myristate, butyl myristate, isopropyl palmitate, ethyl stearate, octyl palmitate, isocetyl isostearate, butyl stearate, ethyl linolate, isopropyl linolate, ethyl olate, isocetyl myristate, isostearyl myristate, isostearyl palmitate, octyldodecyl myristate, isocetyl isostearate, diethyl sebacinate, adipate diisopropyl, isoalkyl neopentanate, tri(capryl, caprylic acid)glyceryl, tri-2-ethylhexanetrimethylolpropane, trimethylolpropane triisostearate, tetra-2-ethylhexanepentapentaerythritol, cetyl caprylate, decyl laurate, hexyl laurate, decyl myristate, myristyl myristate, cetyl myristate, stearyl stearate, decyl olate, cetyl lisinoolate, laurate isostearyl, isotridecyl myristate, isocetyl palmitate, octyl stearate, isocetyl stearate, isodecyl olate, octyldodecyl olate, octyldodecyl linolate, isopropyl isostearate, cetostearyl 2-ethylhexanate, stearyl 2-ethylhexanate, hexyl isostearate, ethyleneglycol dioctanate, ethyleneglycol diolate, propyleneglycol dicaprylate, di(capryl, caprylic acid)propyleneglycol, propyleneglycol dicaprylate, neopentylglycol dicaprylate, neopentylglycol dioctanate, glyceryl tricaprylate, glyceryl triundecylate, glyceryl triisopalmitate, glyceryl triisostearate, neopentanate octyldodecyl, isostearyl octanate, octyl isononate, hexyldecyl neodecanate, octyldodecyl neodecanate, isocetyl isostearate, isostearyl isostearate, octyldecyl isostearate, polyglycerine oleic acid ester, polyglycerine isostearic acid ester, triisocetyl citrate, triisoalkyl citrate, triisooctyl citrate, lauryl lactate, myristyl lactate, cetyl lactate, octyldecyl lactate, triethyl citrate, acetyltriethyl citrate, acetyltributyl citrate, trioctyl citrate, diisostearyl malate, 2-ethylhexyl hydroxystearate, di-2-ethylhexyl succinate, diisobutyl adipate, diisopropyl sebacinate, dioctyl sebacinate, cholesteryl stearate, cholesteryl isostearate, cholesteryl hydroxystearate, cholesteryl olate, dihydrocholesteryl olate, pitsteryl isostearate, pitsteryl olate, 12-isocetyl stealoylhydroxystearate, stearyl 12-stealoylhydroxystearate, and isostearyl 12-stealoylhydroxystearate, but not always limited thereto.

The hydrocarbon oil herein is preferably selected from the group consisting of squalene, liquid paraffin, alpha-olrfinoligomer, isoparaffin, ceresin, paraffin, liquid isoparaffin, polybutene, microcrystalline wax and vaseline, but not always limited thereto.

The silicon oil is preferably selected from the group consisting of polymethylsilicon, methylphenylsilicon, methylcyclopolysiloxane, octamethylpolysiloxane, decamethylpolysiloxane, dodecamethylcyclosiloxane, dimethylsiloxane.methylcetyloxysiloxane copolymer, dimethylsiloxane.methylstealoxysiloxane copolymer, alkyl modified silicon oil and amino modified silicon oil, but not always limited thereto.

The fluorinated oil is preferably perfluoropolyether, but not always limited thereto.

The animal or vegetable oil is preferably selected from the group consisting of avocado oil, almond oil, olive oil, sesame oil, rice bran oil, safflower oil, soybean oil, corn oil, rapeseed oil, apricot kernel oil, palm kernel oil, palm oil, castor oil, sun flower oil, grape seed oil, cottonseed oil, coconut oil, kukuinut oil, wheat germ oil, rice germ oil, shea butter, evening primrose oil, macadamia nut oil, meadowfoam oil, egg yolk oil, beef tallow, hempseed oil, mink oil, orange roughyn oil, jojoba oil, candelilla wax, carnauba wax, liquid lanolin and hydrogenated castor oil, but not always limited thereto.

The moisturizer herein is preferably soluble low-molecular moisturizer, fat soluble low-molecular moisturizer, soluble high-molecular moisturizer or fat soluble high-molecular moisturizer, but not always limited thereto. The soluble low-molecular moisturizer is exemplified by serine, glutamine, sorbitol, mannitol, sodium pyrrolidone-carboxylate, glycerine, propyleneglycol, 1,3-butyleneglycol, ethyleneglycol, polyethyleneglycol B (polymerization degree polypropyleneglycol (polymerization degree n≧2), polyglycerine B (polymerization degree n≧2), lactic acid or lactate, but not always limited thereto. The fat soluble low-molecular moisturizer is exemplified by cholesterol or cholesterol ester, but not always limited thereto. The soluble high-molecular moisturizer is exemplified by carboxyvinylpolymer, polyasparaginate, tragacanth, xanthan gum, methylcellulose, hydroxymethylcellulose, hydroxyethylcellulose, hydroxypropylcellulose, carboxymethylcellulose, soluble chitin, chitosan or dextrin, but not always limited thereto. The fat soluble high-molecular moisturizer is exemplified by polyvinylpyrrolidone.eicosen copolymer, polyvinylpyrrolidone.hexadecen copolymer, nitrocellulose, dextrinfatty acid ester or high-molecular silicon, but not always limited thereto. The emollient is exemplified by long-chain cholesterylester acylglutamate, cholesteryl hydroxystearate, 12-hydroxy stearate, stearic acid, rosinic acid or lanolin fatty acid cholesteryl ester, but not always limited thereto.

The surfactant herein is preferably selected from the group consisting of non-ionic surfactants, anionic surfactants, cationic surfactants and amphoteric surfactants, but not always limited thereto. The non-ionic surfactant is exemplified by self-emulsifying glycerine monostearate, propyleneglycol fatty acid ester, glycerine fatty acid ester, polyglycerine fatty acid ester, sorbitan fatty acid ester, POE (polyoxyethylene)sorbitan fatty acid ester, POE sorbitan fatty acid [symbol] ester, POE glycerine fatty acid ester, POE alkylether, POE fatty acid ester, POE hydrogenated castor oil, POE castor oil, POEPOP (polyoxyethylene.polyoxypropylene) copolymer, POE.POP alkylether, polyether modified silicon, alkanolamide laurate, alkylamineoxide or hydrogenated soybean phospholipid, but not always limited thereto. The anionic surfactant is exemplified by fatty acid soap, alpha-acyl sulfonate, alkyl sulfonate, alkylallyl sulfonate, alkylnaphthalene sulfonate, alkyl sulfate, POE alkylether sulfate, alkylamide sulfate, alkyl phosphate, POE alkyl phosphate, alkylamide phosphate, alkyloylalkyl taurate, N-acyl aminate, POE alkylether carboxylate, alkylsulfosuccinate, sodium alkylsulfoacetate, acylate hydrolyzed collagen peptide salt or perfluoroalkylphosphoester, but not always limited thereto. The cationic surfactant is exemplified by alkyltrimethylammonium chloride, sodium stearyltrimethylammonium, stearyltrimethylammonium bromide, cetostearyltrimethylammonium chloride, di stearyldimethylammonium chloride, stearyldimethylbenzylammonium chloride, behenyltrimethylammonium bromide, benzalconium chloride, diethylaminoethylamide stearate, dimethylaminopropylamide stearate or lanolin derivative fourth grade ammonium salt, but not always limited thereto. The amphoteric surfactant is exemplified by carboxybetaine, amidebetaine, sulfobetaine, hydroxysulfobetaine, amidesulfobetaine, phosphobetaine, aminocarboxylate, imidazoline derivative or amideamine, but not always limited thereto.

The organic and inorganic dyes are preferably selected from the group consisting of inorganic dyes such as silica, silicon dioxide, magnesium silicate, talc, sericite, mica, kaolin, bengala, clay, bentonite, titan enveloped mica, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine, chrome oxide, chrome hydroxide, calamine and conjugates thereof; organic dyes such as polyamide, polyester, polypropylene, polystyrene, polyurethane, vinyl resin, urea resin, phenol resin, fluoro resin, silicon resin, acryl resin, melamine resin, epoxy resin, polycarbonate resin, divinylbenzene.styrene copolymer, silk powder, cellulose, CI pigment yellow and CI pigment orange; and inorganic/organic combined dyes, but not always limited thereto.

The organosol is preferably selected from the group consisting of metal soap such as calcium stearate; alkylphosphometal salt such as sodium zinc cetylate, zinc laurylate and calcium laurylate; acylamino acid polyvalent metal salt such as N-lauroyl-beta-alaninecalcium, N-lauroyl-beta-alaninezinc and N-lauroylglycinecalcium; amidesulfonic acid polyvalent metal salt such as N-lauroyl-taurincalcium and N-palmitoyl-taurincalcium; N-acyl basic amino acid such as N-epsilon-lauroyl-L-lysine, N-epsilon-palmitoyllysine, N-alpha-palmitoylolnitine, N-alpha-lauroylalrginine and N-alpha-hydrogenated beef tallow fatty acid acylarginine; N-acylpolypeptide such as N-lauroylglycylglycine; alpha-amino fatty acid such as alpha-aminocaprylic acid and alpha-aminolauric acid; polyethylene; polypropylene; nylon; polymethylmethacrylate; polystylene; divinylbenzene/styrene copolymer; and ethylene tetrafluoride, but not always limited thereto.

The UV absorbent herein is preferably selected from the group consisting of paraminobenzoate, ethyl paraminobenzoate, amyl paraminobenzoate, octyl paraminobenzoate, ethyleneglycol salicylate, phenyl salicylate, octyl salicylate, benzyl salicylate, butylphenyl salicylate, homomethyl salicylate, benzyl cinnamate, 2-etoxyethyl parametoxy cinnamate, octyl parametoxycinnamate, mono-2-ethylhexaneglyceryl diparametoxycinnamate, isopropyl parametoxycinnamate, diisopropyl.diisopropyl cinnamate ester mixture, urocanic acid, ethyl urokanate, hydroxymetoxybenzophenone, hydroxymetoxybenzophenonesulfonate and salts thereof, dihydroxymetoxybenzophenone, sodium dihydroxymetoxybenzophenone disulfonate, dihydroxybenzophenone, tetrahydroxybenzophenone, 4-tert butyl-4′-metoxydibenzoylmethane, 2,4,6-trianilino-p-(carbo-2′-ethylhexyl-1′-oxy)-1,3,5-triazine and 2-(2-hydroxy-5-methylphenyl)benzotriazol, but not always limited thereto.

The bactericide herein is preferably selected from the group consisting of hinokitiol, trichloric acid, trichlorohydroxydiphenylether, chlorohexidingluconate, phenoxyethanol, resorcin, isopropylmethylphenol, azulene, salicylic acid, zinc philithion, benzalconium chloride, phtosensitizer 301, sodium mononitroguaiacol and undecylenic acid, but not always limited thereto.

The antioxidant herein is preferably butylhydroxyanisole, propyl galate or erythorbic acid, but not always limited thereto.

The pH regulator herein is preferably citric acid, sodium citrate, malic acid, sodium malate, fumaric acid, sodium fumarate, succinic acid, sodium succinate, sodium hydroxide or sodium phosphate dibasic, but not always limited thereto.

The alcohol herein is higher alcohol such as cetyl alcohol, but not always limited thereto.

The constituents are not limited to the above ingredients, and any ingredient can be added as long as it cannot damage the object and effect of the present invention. At this time, preferable ratio of the additive is 0.01-5 weight % by the total weight and 0.01˜3 weight % is more preferred, but not always limited thereto.

The cosmetic composition of the present invention can be formulated in the form of solution, emulsion or viscous mixture, but not always limited thereto. The cosmetic composition of the present invention can include, in addition to the said active ingredient, any conventional ingredients generally used in cosmetics, for example such additives and carriers as stabilizers, solubilizers, vitamins, pigments and flavors, but not always limited thereto.

The cosmetic composition of the present invention can be formulated in any form that can be accepted in the art, which is exemplified by emulsion, cream, lotion, pack, foundation, lotion, essence and hair composition, but not always limited thereto. Particularly, the cosmetic composition of the present invention can be prepared in the form of skin lotion, skin softener, skin toner, astringent, lotion, milk lotion, moisture lotion, nutritive lotion, massage cream, nutritive cream, moisture cream, hand cream, foundation, essence, nutritive essence, pack, soap, cleansing foam, cleansing lotion, cleansing cream, body lotion and body cleanser, but not always limited thereto.

In the case that the cosmetic composition of the present invention is formulated as paste, cream or gel, the proper carrier can be selected from the group consisting of animal oil, vegetable oil, wax, paraffin, starch, trakind, cellulose derivative, polyethylene glycol, silicon, bentonite, silica, talk and zinc oxide, but not always limited thereto.

In the case that the cosmetic composition of the present invention is formulated as powder or spray, the proper carrier can be selected from the group consisting of lactose, talc, silica, aluminum hydroxide, calcium silicate and polyamide powder, and in particular if the composition of the present invention is formulated as spray, a propellant such as chlorofluorohydrocarbon, propane/butane or dimethyl ether can be additionally included, but not always limited thereto.

In the case that the cosmetic composition of the present invention is formulated as liquid or emulsion, the proper carrier can be selected from the group consisting of solvent, solubilizer and emulsifier, which is exemplified by water, ethanol, isopropanol, ethyl carbonate, ethyl acetate, benzyl alcohol, benzyl benzoate, propylene glycol, 1,3-butyl glycol oil, glycerol aliphatic ester, polyethylene glycol and fatty acid ester of sorbitan, but not always limited thereto.

In the case that the cosmetic composition of the present invention is formulated as suspension, the proper carrier can be selected from the group consisting of liquid diluent such as water, ethanol or propylene glycol; suspending agent such as ethoxylated isostearyl alcohol, polyoxyethylene sorbitol ester and polyoxyethylene sorbitan ester; microcrystalline cellulose; aluminum methahydroxide; bentonite; agar; and tragacanth, but not always limited thereto.

In the case that the cosmetic composition of the present invention is formulated as surfactant-containing cleansing, the proper carrier can be selected from the group consisting of aliphatic alcohol sulfate, aliphatic alcohol ether sulfate, sulfosuccinic monoester, isethionate, imidazolinum derivative, methyltaurate, sarcosinate, fatty acid amide ether sulfate, alkyl amidobetain, aliphatic alcohol, fatty acid glyceride, fatty acid diethanolamide, vegetable oil, lanolin derivative and ethoxylated glycerol fatty acid ester, but not always limited thereto.

### ADVANTAGEOUS EFFECTIVENESS

Docosahexaenoyl lysophosphatidylamine of the present invention inhibits NO generation, has no cytotoxicity, and suppresses peritonitis in vivo at a low concentration, so that it can be effectively used as an anti-inflammatory composition for prevention and treatment of inflammatory disease.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Intracellular Anti-Inflammatory Effect of Docosahexaenoyl Lysophosphatidylcholine

Macrophages are known to play an important role in inflammatory response and host defense. When macrophages are activated by lipopolysaccharide (LPS), a huge amount of NO (nitric oxide) is generated, which is mediated by inducible nitric oxide synthase (iNOS).

Based on that, the present inventors observed how unsaturated fatty acid containing lysophosphatidylcholine (lysoPC) affected the generation of NO, the inflammation mediator, induced by LPS in RAW 264.7 cells. Particularly, RAW 264.7 cells (5×10⁴/well) were pre-cultured with each polyunsaturated lysophosphatidylcholine; linoleoyl lysophosphatidylcholine, arachidonoyl lysophosphatidylcholine and docosahexaenoyl lysophosphatidylcholine [prepared by the co-treatment with diacyl phosphatidylcholine (Avanti Polar Lipids, Alabaster, Ala., USA) and phospholipase A₂ (PLA₂, Sigma-Aldrich Corp, St. Louis, Mo., USA) (Croset, M. et al., Biochem. J. 2000, 345, 61-67; Adachi, J. et al., Kobe. J. Med. Sci. 2006, 52, 127-140)] (0˜60 uM) at 37° C. for 2 hours. Then, LPS (1 μg/ml) was added thereto, followed by culture for 20 hours under the same conditions as the above. Griess reagent [1% sulfanilamide, 0.1% N-(1-naphtyl)ethylenediamine dihydrochloride in 2.5% H₃PO₄] was added to the supernatant of the culture solution, followed by reaction at room temperature for 15 minutes. Then, OD₅₄₀ was measured to measure NO generation (Green L C, et al., Anal Biochem. 1982 October; 126(1):131-8).

As a result, as shown in FIG. 1B, arachidonoyl lysophosphatidylcholine inhibited NO generation induced by LPS at the concentration of 60 uM to some degree but the inhibition was not greater than 20%. In the meantime, docosahexaenoyl lysophosphatidylcholine demonstrated significant inhibitory effect at the low concentration of 12 uM. At this time, EC₅₀ was 18.2±2.1 uM. On the other hand, linoleoyl lysophosphatidylcholine did not show any significant inhibitory effect at the concentration of up to 60 uM (FIG. 1B).

### Example 2

### Cytotoxicity of Docosahexaenoyl Lysophosphatidylcholine

MTT assay was performed to investigate cytotoxicity of three kinds of polyunsaturated lysophosphatidylcholines. Particularly, RAW 264.7 cells were cultured in DMEM containing penicillin (100 U/ml), streptomycin (100 U/ml) and heat-inactivated FBS (10% v/v) in 37° C., 5% CO₂ incubator. The cells (6-13 passage) were recovered and distributed into a 96-well plate (5×10⁴/well), followed by culture. After treating each polyunsaturated lysophosphatidylcholine (0˜60 uM), the plate stood for 2 hours. The cells were cultured under the same conditions as the above in the presence of LPS (1 μg/ml) for 20 hours. Then, cell death rate was investigated by comparing OD₅₇₀ and OD₆₉₀ based on MTT(3-(4,5-dimethylthiazol-2yl)-2,5-diphenyl-2H-tetrazolium bromide) method (Lin W W. et al., Br. J. Pharmacol 1998; 125: 1601-1609; Mosmann, T., J. Immunol. Methods 1983; 65:55-63).

AS a result, as shown in FIG. 1A, arachidonoyl lysophosphatidylcholine showed rather significant dose-dependent cytotoxicity, while linoleoyl lysophosphatidylcholine or docosahexaenoyl lysophosphatidylcholine did not show significant cytotoxicity (FIG. 1A).

Therefore, No generation inhibition by arachidonoyl lysophosphatidylcholine could be presumed to be resulted from cytotoxicity, but, NO generation inhibited by docosahexaenoyl lysophosphatidylcholine was not related to cytotoxicity. That is, docosahexaenoyl lysophosphatidylcholine was confirmed to have selective NO generation inhibitory effect.

### Example 3

### In Vivo Anti-Inflammatory Effect of Docosahexaenoyl Lysophosphatidylcholine Via Intravenous Injection

### <3-1> Inhibitory Effect of Lysophosphatidylcholine on Zymosan A Mediated Plasma Leakage

5-Lipoxygenase (5-LOX) plays an important role in the inflammation mediator leukotriene generation from arachidonic acid. So, it was reported that inflammatory response could be reduced by the inhibition of 5-LOX. It is also known that zymosan induced peritonitis is mediated by leukotriene generated by 5-LOX (RaoTS, et al., J Pharmacol Exp Ther. 1994 June; 269(3):917-25; Doherty N S, et al., Prostaglandins. 1985 November; 30(5):769-89).

To investigate inhibitory effect of docosahexaenoyl lysophosphatidylcholine on peritonitis induced by zymosan, the present inventors induced inflammation in abdominal cavity by injecting zymosan A (saccharomyces cerevisiae) (Sigma-Aldrich Corp, St. Louis, Mo., USA), and then administered lysophosphatidylcholine, followed by evaluation of anti-inflammatory effect. Particularly, SPF mice (6 weeks, male) were adapted for 12 hours and then raised in an animal laboratory at College of Pharmacy under 12/12 hour dark/light cycle. Then, the mice were used for intra-abdominal inflammatory response experiment. First, each experimental group was composed of 5˜10 mice, to which 10˜500 μg/kg of each lysophosphatidylcholine was injected through tail vein. The control group was injected with 50˜100 ul of saline. 30 minutes later, 0.5% Evans blue dye solution (0.2 ml) was injected through tail vein and then 100 ul of zymosan A (1 mg/ml) dissolved in saline was injected into abdominal cavity. 30 minutes after the administration, the mice were anesthetized lightly with ether or fluorine, followed by neck dissection. 4 ml of ice-cold PBS was injected into abdominal cavity and peritoneal fluid was recovered by using a plastic syringe, followed by centrifugation (3,000 rpm, 10 minutes). Supernatant was obtained from the centrifugation, with which exudation of Evans blue was measured by spectrophotometer at 620 nm to evaluate anti-inflammatory effect (Arita M, et al., Proc Natl Acad Sci USA. 2005 May 24; 102(21):7671-6; Takenaka M, et al., Biol Pharm Bull. 2005 July; 28(7):1291-3).

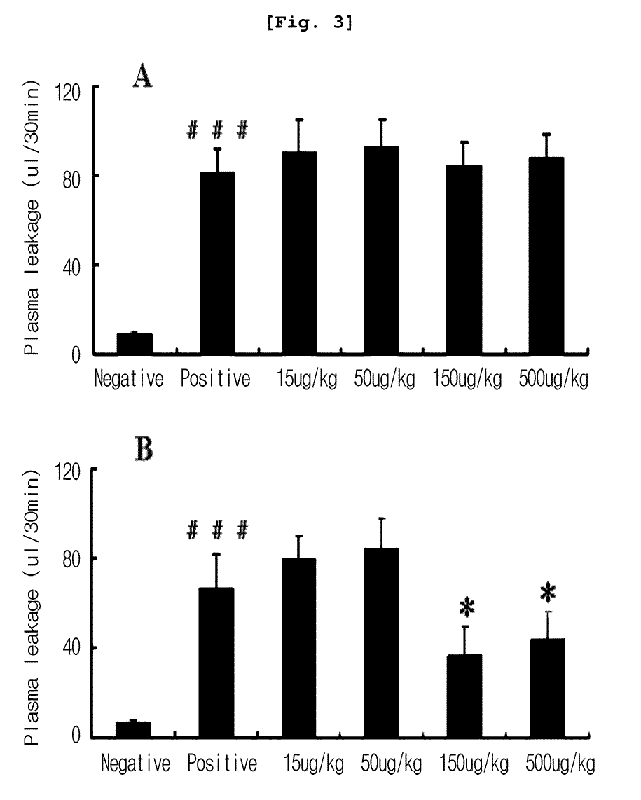

As a result, as shown in FIG. 2, docosahexaenoyl lysophosphatidylcholine demonstrated significant inflammation inhibitory effect at the concentration of 15 μg/kg, indicating that docosahexaenoyl lysophosphatidylcholine inhibited zymosan induced plasma leakage dose-dependently up to the concentration of 500 μg/kg (FIG. 2). In the meantime, arachidonoyl lysophosphatidylcholine reduced plasma leakage significantly at the concentration of 50 μg/kg, which was more peculiar at the concentration of 150 μg/kg but this effect was not increased any more even when the concentration was increased to 500 μg/kg. On the other hand, linoleoyl lysophosphatidylcholine did not show inhibitory effect up to the concentration of 500 μg/kg (FIG. 3).

Therefore, it was confirmed that docosahexaenoyl lysophosphatidylcholine had excellent inhibitory effect on zymosan A induced plasma leakage, compared with other lysophosphatidylcholines.

### <3-2> Time-Dependent Inhibitory Effect of Lysophosphatidylcholine on Zymosan A Induced Plasma Leakage

The present inventors measured time-dependent anti-inflammatory effect (inhibitory effect on zymosan A induced plasma leakage) of each lysophosphatidylcholine. Particularly, docosahexaenoyl lysophosphatidylcholine (50 μg/kg) was injected to mice through tail vein at different time intervals (15 minutes—120 minutes; 10 minutes, 30 minutes, 60 minutes and 120 minutes before zymosan A administration). After administrating zymosan A (100 mg/kg) via intraperitoneal injection, Evans blue was treated, followed by measuring plasma leakage in peritoneal fluid.

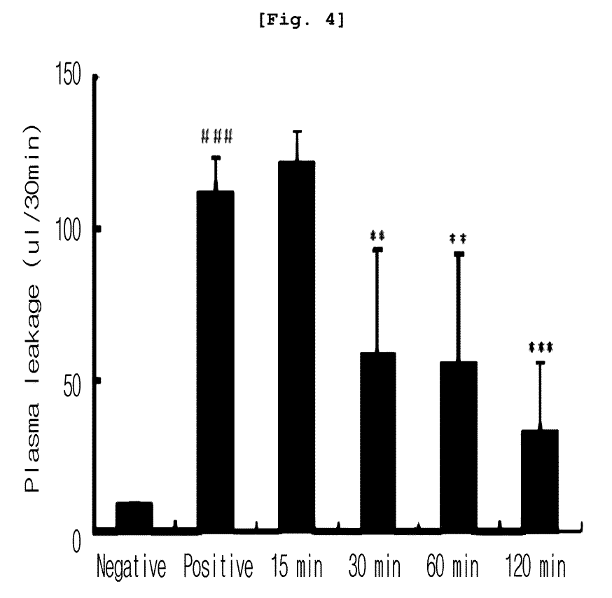

As a result, as shown in FIG. 4, when docosahexaenoyl lysophosphatidylcholine was administered 10 minutes before zymosan A injection, the inhibitory effect was hardly observed. But when it was administered 30 minutes or 60 minutes before zymosan A injection, the inhibitory effect was significant. In the meantime, when it was administered 120 minutes before zymosan A injection, the inhibitory effect was slightly increased (FIG. 4).

### <3-3> Inhibitory Effect of Docosahexaenoic Acid or the Peroxide Thereof on Zymosan A Induced Plasma Leakage

In Example <2-1>, where the compound was administered long before (120 minutes earlier) zymosan A injection, it was presumed that there were other mechanisms involved. So, when injection interval was longer, it seemed that at least two different inhibitory mechanisms were involved; short-term inhibitory effect on zymosan A induced plasma leakage and long-term inhibitory effect on zymosan A induced plasma leakage. The short-term inhibitory effect, presumably due to the direct inhibition of 5-lipoxygenase, of docosahexaenoyl lysophosphatidylcholine on zymosan A induced plasma leakage may be less likely, since leukotriene C-induced plasma leakage was prevented by docosahexaenoyl lysophosphatidylcholine.

To investigate whether the said long-term effect was attributed to metabolites generated by metabolism or not, docosahexaenoyl lysophosphatidylcholine, docosahexaenoic acid and 17-HPDHA lysophosphatidylcholine (17(S)-hydroperoxy-4,7,10,13,15,19-docosahexaenoyl lysophosphatidylcholine) were injected into abdominal cavity, followed by observation in plasma leakage in peritoneal fluid. Relations of docosahexaenoic acid and 17-HPDHA lysophosphatidylcholine with docosahexaenoyl lysophosphatidylcholine are as follows:

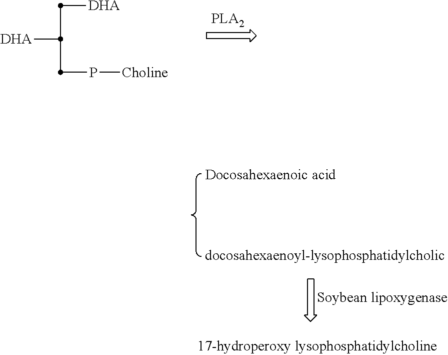

Particularly, docosahexaenoyl lysophosphatidylcholine (50 μg/kg), docosahexaenoic acid (50 μg/kg) or 17-HPDHA lysophosphatidylcholine (17(S)-hydroperoxy-4,7,10,13,15,19-docosahexaenoyl lysophosphatidylcholine) were compared. Soybean LOX-1 (200 units/ml) was added in borax buffer (50 mM, pH 9.0) containing docosahexaenoyl lysophosphatidylcholine (20 uM), followed by reaction at 25° C. for 30 minutes, which was passed through C₁₈ column (2×1 cm) and eluted with methanol (Huang L S, et al., J Agric Food Chem. 2008 Sep. 10; 56(17):7808-14)] (50 μg/kg) was treated to mice 30 minutes before zymosan A injection. 30 minutes later, plasma leakage in peritoneal fluid was measured.

As a result, as shown in FIG. 5, docosahexaenoic acid did not show any significant inhibitory effect on plasma leakage. Meanwhile, docosahexaenoyl lysophosphatidylcholine peroxide demonstrated a greater inhibitory effect, compared to docosahexaenoyl lysophosphatidylcholine (FIG. 5).

Therefore, it was confirmed that inhibitory effect of docosahexaenoyl lysophosphatidylcholine on plasma leakage was resulted from oxygenation of docosahexaenoyl lysophosphatidylcholine.

### Example 4

### In Vivo Anti-Inflammatory Effect of Docosahexaenoyl Lysophosphatidylcholine Via Intraperitoneal Injection

The present inventors intraperitoneally injected docosahexaenoyl lysophosphatidylcholine or 17-HPDHA lysophosphatidylcholine (docosahexaenoyl lysophosphatidylcholine oxide) into mice at the concentration of 15˜150 μg/kg. The control group was injected with 50˜100 ul of saline. 2 Hours later, 0.5% Evans Blue dye solution (0.2 ml) was injected through tail vein, followed by intraperitoneal injection of 100 ul of zymosan A (1 mg/ml) dissolved in saline. 30 minutes later, the mice were slightly anesthetized with ether or fluorine, followed by neck dissection. 4 ml of ice-cold PBS was injected into abdominal cavity and peritoneal fluid was recovered by using a plastic syringe, followed by centrifugation (3,000 rpm, 10 minutes). Exudation of Evans blue was measured to evaluate anti-inflammatory effect.

As a result, as shown in FIG. 6, docosahexaenoyl lysophosphatidylcholine intraperitoneally injected demonstrated significant inhibitory effect at the concentration of 15 μg/kg or higher. Docosahexaenoyl lysophosphatidylcholine inhibited zymosan induced plasma leakage dose-dependently up to the concentration of 150 μg/kg (FIG. 6, open). In addition, 17-HPDHA lysophosphatidylcholine also inhibited plasma leakage, which was significant at the concentration of 15 μg/kg and dose-dependent up to the concentration of 150 μg/kg. In comparison, 17-HPDHA lysophosphatidylcholine was somewhat more effective than docosahexaenoyl lysophosphatidylcholine, suggesting that docosahexaenoyl lysophosphatidylcholine may be converted to bioactive metabolites through 17-HPDHA derivative.

Therefore, it was confirmed that docosahexaenoyl lysophosphatidylcholine or 17-HPDHA lysophosphatidylcholine effectively inhibited zymosan A induced plasma leakage by intraperitoneal injection even at low concentrations, suggesting that they had excellent anti-inflammatory effect.

### Example 5

### In Vivo Anti-Inflammatory Effect of Docosahexaenoyl Lysophosphatidylethanolamine Via Intravenous Injection

The present inventors intravenously injected docosahexaenoyl lysophosphatidylethanolamine into mice at the concentration of 15˜150 μg/kg. 30 Min later, 100 ul of zymosan A(1 mg/ml) was treated thereto to induce inflammation in abdominal cavity. After administering 0.5% Evans blue, plasma leakage in peritoneal fluid was measured.

As a result, as shown in FIG. 7, docosahexaenoyl intravenously injected demonstrated significant inhibitory effect at the concentration of 50 μg/kg or higher. docosahexaenoyl lysophosphatidylethanolamine inhibited zymosan A induced plasma leakage dose-dependently up to the concentration of 150 μg/kg (FIG. 7, open).

The Manufacturing Examples for the composition of the present invention are described hereinafter.

### Manufacturing Example 1

### Preparation of Pharmaceutical Formulations

<1-1> Preparation of powders

| | |
|---|---|
| Docosahexaenoyl lysophosphatidylcholine | 20 mg |
| Lactose | 20 mg |

Powders were prepared by mixing all the above components, which were filled in airtight packs according to the conventional method for preparing powders.

### <1-2> Preparation of Tablets

| | |
|---|---|
| Docosahexaenoyl lysophosphatidylcholine | 10 mg |
| Corn starch | 100 mg |
| Lactose | 100 mg |
| Magnesium stearate | 2 mg |

Tablets were prepared by mixing all the above components by the conventional method for preparing tablets.

### <1-3> Preparation of Capsules

| | | |
|---|---|---|
| Docosahexaenoyl lysophosphatidylcholine | 10 | mg |
| Corn starch | 3 | mg |
| Lactose | 14.8 | mg |
| Magnesium stearate | 0.2 | mg |

Capsules were prepared by mixing all the above components, which were filled in gelatin capsules according to the conventional method for preparing capsules.

### <1-4> Preparation of Liquid Preparations

| | | |
|---|---|---|
| Docosahexaenoyl lysophosphatidylcholine | 20 | mg |
| Isomerized sugar | 10 | g |
| Mannitol | 5 | g |

| | |
|---|---|
| Purified water | proper amount |

All the above components were dissolved in purified water. After adding lemon flavor, total volume was adjusted to be 100 Ml by adding purified water. Liquid formulations were prepared by putting the mixture into brown bottles and sterilizing thereof by the conventional method for preparing liquid formulations.

### <1-5> Preparation of Injectable Solutions

| | |
|---|---|
| Docosahexaenoyl lysophosphatidylcholine | 10 mg/Ml |
| Weak HCl BP | until pH 7.6 |
| Injectable NaCl BP | up to 1 Ml |

Docosahexaenoyl lysophosphatidylcholine was dissolved in proper volume of injectable NaCl BP. pH of the prepared solution was regulated as 7.6 by using weak HCl BP. The volume was adjusted by using injectable NaCl BP. The solution was well mixed and filled in 5 Ml type I transparent glass ampoules. The ampoules were sealed by melting the glass of opening, followed by UV sterilization.

### Manufacturing Example 2

### Preparation of Foods

Foods containing extracts or fractions of the present invention were prepared as follows.

### <2-1> Preparation of Flour Food

0.005˜0.5 weight part of docosahexaenoyl lysophosphatidylcholine of the present invention was added to flour. Health enhancing foods such as bread, cake, cookies, crackers and noodles were prepared with the flour mixture according to the conventional method.

### <2-2> Preparation of Dairy Products

0.01˜0.1 weight part of docosahexaenoyl lysophosphatidylcholine of the present invention was added to milk. Health enhancing dairy products such as butter and ice cream were prepared with the milk mixture according to the conventional method.

### <2-3> Preparation of Sun-Sik

Brown rice, barley, glutinous rice and Yulmu (Job's tears) were gelatinized according to the conventional method, dried and pulverized to obtain 60-mesh powders.

Black soybean, black sesame and wild sesame were steamed and dried according to the conventional method and pulverized to obtain 60-mesh powders.

Docosahexaenoyl lysophosphatidylcholine of the present invention was concentrated under reduced pressure, spray-dried and pulverized to obtain 60-mesh dry powders.

Sun-Sik was prepared by mixing the dry powders of the grains, seeds and docosahexaenoyl lysophosphatidylcholine according to the below ratio.

Grains (brown rice: 30 weight part, Yulmu: 15 weight part, barley: 20 weight part),

Seeds (wild sesame: 7 weight part, black soybean: 8 weight part, black sesame: 7 weight part),

Dry powders of docosahexaenoyl lysophosphatidylcholine (0.1 weight part),

Ganoderma lucidum (0.5 weight part),

Rehmannia glutinosa (0.5 weight part)

### <2-4> Preparation of Health Food

| docosahexaenoyl lysophosphatidylcholine | 0.5 | mg |
|---|---|---|
| Vitamin complex | proper amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Minerals | proper amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Potassium phosphate dibasic | 55 | mg |
| Potassium citrate | 90 | mg |
| Calcium carbonate | 100 | mg |
| Magnesium chloride | 24.8 | mg |

Vitamins and minerals were mixed according to the preferable composition rate for health food. However, the composition rate can be adjusted. The constituents were mixed according to the conventional method for preparing health food and then the composition for health food was prepared according to the conventional method.

### Manufacturing Example 3

### Preparation of Health Beverages

| | | |
|---|---|---|
| Docosahexaenoyl lysophosphatidylcholine | 1 | mg |
| Citric acid | 100 | mg |
| Oligosaccharide | 100 | mg |
| Maesil (Prunus mume) Extract | 2 | mg |
| Taurine | 100 | mg |
| Purified water | up to 500 | Ml |

The above constituents were mixed according to the conventional method for preparing health beverages. The mixture was heated at 85° C. for 1 hour with stirring and then filtered. The filtrate was loaded in 1 liter sterilized containers, which were sealed and sterilized again, stored in a refrigerator until they would be used for the preparation of a composition for health beverages.

The constituents appropriate for favorite beverages were mixed according to the preferred mixing ratio but the composition ratio can be adjusted according to regional and national preferences, etc.

### Manufacturing Example 4

### Preparation of Cosmetics

### <4-1> Preparation of Emulsified Cosmetics

Emulsified cosmetics were prepared according to the composition shown in Table 1. The method for the preparation is as follows.

1) heating the mixture comprising the raw materials 1-9 at 65-70° C.;

2) adding the raw material 10 to the mixture of step 1);

3) dissolving the mixture comprising the raw materials 11-13 by heating at 65-70° C.;

4) slowly adding the mixture of step 2) during performing step 3), followed by emulsification at 8,000 rpm for 2-3 minutes;

5) dissolving the raw material 14 in water and then adding the solution to the mixture of step 4), followed by emulsification for 2 minutes;

6) weighing the raw materials 15-17, which were added to the mixture of step 5), followed by emulsification for 30 seconds; and

7) degassing the mixture of step 6) finished with the emulsification process and then cooling thereof at 25-35° C. to give emulsified cosmetics.

TABLE 1
Compositions of emulsified formulations 1, 2 and 3

| | | Emulsified | Emulsified | Emulsified |
|---|---|---|---|---|
| | | formulation | formulation | formulation |
| Composition | | 1 | 2 | 3 |
| 1 | Stearic acid | 0.3 | 0.3 | 0.3 |
| 2 | Stearyl alcohol | 0.2 | 0.2 | 0.2 |
| 3 | Glyceryl monostearate | 1.2 | 1.2 | 1.2 |
| 4 | Wax | 0.4 | 0.4 | 0.4 |
| 5 | Polyoxyethylenesorbitan | 2.2 | 2.2 | 2.2 |
| | monolauric acid ester | | | |
| 6 | Paraoxybenzoic acid | 0.1 | 0.1 | 0.1 |
| | methyl | | | |
| 7 | Paraoxybenzoic acid | 0.05 | 0.05 | 0.05 |
| | propyl | | | |
| 8 | Cetyl ethyl hexanoate | 5 | 5 | 5 |
| 9 | Triglyceride | 2 | 2 | 2 |
| 10 | Cyclomethicone | 3 | 3 | 3 |
| 11 | Distilled water | to 100 | to 100 | to 100 |
| 12 | Concentrated Glycerin | 5 | 5 | 5 |
| 13 | Triethanolamine | 0.15 | 0.15 | 0.15 |
| 14 | Polyacrylic acid | 0.12 | 0.12 | 0.12 |
| | polymer | | | |
| 15 | Pigment | 0.0001 | 0.0001 | 0.0001 |
| 16 | Flavor | 0.10 | 0.10 | 0.10 |
| 17 | Docosahexaenoyl | 0.0001 | 0.003 | 0.01 |
| | lysophosphatidylcholine | | | |

### <4-2> Preparation of Solubilized Cosmetics

Solubilized cosmetics were prepared according to the composition shown in Table 2. The method for the preparation is as follows.

1) adding the raw materials 2-6 to the raw material 1 (purified water), which were dissolved by using Agi-mixer;

2) adding the raw materials 8-11 to the raw material 7 (alcohol) and completely dissolved; and

3) slowly adding the mixture of step 2) to the mixture of step 1), followed by solubilization.

TABLE 2
Compositions of solubilized formulations 1, 2 and 3

| | | solubilized | solubilized | solubilized |
|---|---|---|---|---|
| | | formulation | formulation | formulation |
| Composition | | 1 | 2 | 3 |
| 1 | Purified water | to 100 | to 100 | to 100 |
| 2 | Concentrated | 3 | 3 | 3 |
| | glycerin | | | |
| 3 | 1,3-butyleneglycol | 2 | 2 | 2 |
| 4 | EDTA-2Na | 0.01 | 0.01 | 0.01 |
| 5 | Pigment | 0.0001 | 0.0002 | 0.0002 |
| 6 | Docosahexaenoyl | 0.1 | 5 | 5 |
| | lysophosphatidyl- | | | |
| | choline | | | |
| 7 | Alcohol (95%) | 8 | 8 | 8 |
| 8 | Paraoxybenzoic | 0.1 | 0.1 | 0.1 |
| | acid methyl | | | |
| 9 | Polyoxyethylene | 0.3 | 0.3 | 0.3 |
| | hydrogenated ester | | | |
| 10 | Flavor | 0.15 | 0.15 | 0.15 |
| 11 | Cyclomethicone | — | — | 0.2 |

### INDUSTRIAL APPLICABILITY

As explained hereinbefore, docosahexaenoyl lysophosphatidylamine can be effectively used for the development of a therapeutic agent for inflammatory disease, inflammatory disease improving health food and anti-inflammatory cosmetics.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for inhibiting inflammation containing the step of administering docosahexaenoyl lysophosphatidylamine to a subject with inflammation.

2. The method for inhibiting inflammation according to claim 1, wherein the docosahexaenoyl lysophosphatidylamine is selected from the group consisting of docosahexaenoyl lysophosphatidylcholine, docosahexaenoyl lysophosphatidylethanolamine and 2-docosahexaenoyl lysophosphatidylcholine.

3. The method for inhibiting inflammation according to claim 1, wherein the method for inhibiting inflammation comprises a method for treating inflammatory disease by administering a pharmaceutically effective dose of docosahexaenoyl lysophosphatidylamine to a subject with inflammatory disease.

4. The method for treating inflammatory disease according to claim 3, wherein the docosahexaenoyl lysophosphatidylamine is selected from the group consisting of docosahexaenoyl lysophosphatidylcholine, docosahexaenoyl lysophosphatidylethanolamine and 2-docosahexaenoyl lysophosphatidylcholine.

5. The method for treating inflammatory disease according to claim 3, wherein the inflammatory disease is selected from the group consisting of asthma, peritonitis, gastritis, enteritis, arthritis, nephritis, hepatitis and degenerative disease.

6. The method for inhibiting inflammation according to claim 1, wherein the method for inhibiting inflammation comprises a method for preventing inflammatory disease by administering a pharmaceutically effective dose of docosahexaenoyl lysophosphatidylamine to a subject.

7. The method for preventing inflammatory disease according to claim 6, wherein the docosahexaenoyl lysophosphatidylamine is selected from the group consisting of docosahexaenoyl lysophosphatidylcholine, docosahexaenoyl lysophosphatidylethanolamine and 2-docosahexaenoyl lysophosphatidylcholine.

8. The method for preventing inflammatory disease according to claim 6, wherein the inflammatory disease is selected from the group consisting of asthma, peritonitis, gastritis, enteritis, arthritis, nephritis, hepatitis and degenerative disease.
